(19) United States
(12) Patent Application Publication
(10) Pub. No.: US 2023/0346998 A1
(43) Pub. Date: Nov. 2, 2023

(54) MISTY PLASMA DIELECTRIC BARRIER DISCHARGE PLASMA SYSTEM FOR DISINFECTION

(71) Applicant: UNIVERSITY OF SOUTH CAROLINA, COLUMBIA, SC (US)

(72) Inventors: MALIK M. TAHIYAT, COLUMBIA, SC (US); TANVIR I. FAROUK, COLUMBIA, SC (US); SHAMIA HOQUE, COLUMBIA, SC (US); TRACI TESTERMAN, COLUMBIA, SC (US)

(21) Appl. No.: 18/302,329

(22) Filed: Apr. 18, 2023

Related U.S. Application Data
(60) Provisional application No. 63/335,482, filed on Apr. 27, 2022.

(51) Int. Cl. A61L 2/14; A61L 2/26

(52) U.S. Cl. CPC A61L 2/14; A61L 2/26; A61L 2202/11; A61L 2202/16

(57) ABSTRACT

Presently disclosed subject matter relates to application of dielectric barrier discharge in misty plasma systems for disinfection of surfaces and equipment in health settings. A system and corresponding and/or associated methodology is provided for planar dielectric barrier discharge (DBD) device technology. Per one exemplary embodiment, a DBD device is provided with an electrically insulated annular flow channel built through the planar powered electrode. The discharge medium is air saturated with water vapor that flows through the annulus and forms a stagnation discharge plane between the powered dielectric and the substrate. Application on Escherichia (E.) coli and Bacillus (B.) atrophaeus spores on agar and filter papers show that usage of a flowing humid discharge medium has a ˜40% higher efficacy than either a static or a dry flowing medium. The substrate turns acidic potentially due to the formation of reactive oxygen and nitrogen species. In the presence of water vapor, pH can be decreased further due to formation of hydrogen peroxide from recombination of OH radicals formed in the plasma.

### PRIORITY CLAIM

The present application claims the benefit of priority of U.S. Provisional Patent Application No. 63/335,482, titled Misty Plasma Dielectric Barrier Discharge Plasma System For Disinfection, filed Apr. 27, 2022, and which is fully incorporated herein by reference for all purposes.

### BACKGROUND OF THE PRESENTLY DISCLOSED SUBJECT MATTER

The presently disclosed subject matter deals with a system and corresponding and/or associated methodology for planar dielectric barrier discharge (DBD) device technology, and more particularly to a DBD device with an electrically insulated annular flow channel built through the planar powered electrode.

Approximately 1.7 million healthcare associated infections occur each year in the U.S., killing 98,000 patients. COVID-19 has revealed further weaknesses in the preparedness of healthcare facilities, which lacked the ability to adequately disinfect existing personal protective equipment (PPE), exacerbating an already precarious situation. Chemical germicides, UV light etc. approaches have proven insufficient. Recently, application of dielectric barrier discharge (DBD) on biological substrates has been a topic of intense research.

The application of cold atmospheric pressure plasmas such as dielectric barrier discharge (DBD), which are part of nonthermal gaseous plasmas (NTGP), has been a topic of intense research in biological, medical, and environmental fields in recent years [1-13]. Blood coagulation, ablation, incision and cosmetic applications are four broad categories in the medical field where gaseous plasmas are applied currently [14]. NTGP has been shown to have the ability to disinfect, heal and decontaminate wounds in a relatively short time through the production of reactive oxygen and nitrogen species (RONS) without affecting mammalian cells or creating any pollutant as byproduct.

As a disinfectant for environmental surfaces NTGP has shown promise with its efficiency apparently dependent on the microorganism type, the type of discharge medium and the plasma power density [14, 15]. Application of dielectric barrier discharge (DBD) plasma treatment to bacterial cells caused cell wall damage, cytoplasmic damage and chromosome leakage [15]. It has also been shown to damage influenza [32], hepatitis B [26] and herpes virus [13]. The evidence that environmental contamination with health care associated pathogens pose a significant risk for patient to patient transmission is overwhelming with approximately 1.7 million health care-associated infections occur each year in the U.S., killing 98,000 patients [16]. Current methods of reducing contamination levels of environmental surfaces in hospital rooms include routine and terminal disinfection with chemical germicides, and more recently with the application of ‘no touch’ method utilizing ultraviolet (UV) light [17] or aerosolized/vaporized hydrogen peroxide gas plasma [18, 19] as well as ozone or ethylene oxide. However, studies have shown that decontamination efforts with the prescribed methods are often insufficient, leaving microbial contamination present on surfaces [20, 21].

While use of ‘no touch’ methods have improved decontamination efficacy, a significant disadvantage of the use of this approach is that it can only be used for terminal room disinfection requiring removal of patients and health care personnel from the area [22, 23]. Additionally, COVID-19 has revealed further weaknesses in the preparedness of health care facilities, which lacked the ability to adequately disinfect existing personal protective equipment (PPE), exacerbating an already precarious situation.

Thus, nonthermal based plasma technologies have the potential of bringing about a novel approach for rapid, on site and mobile disinfection approach.

For low-temperature plasmas (LTP) oxygen radicals generated in the discharge medium consisting of helium and oxygen appeared to play a primary role in the inactivation of E. coli and B. subtilis [24] [25]. Increasing exposure time to atmospheric pressure DBD caused exponential decrease of the DNA count of HBV (Hepatitis B virus). The interaction of RONS produced in the DBD with the lipids and proteins in the outer capsid of HBV is proposed as a probable deactivation mechanism [26]. Kalghatgi et al. showed that reactive species: both charged and neutrals produced by DBD plasmas can be ‘tuned’ by varying the discharge parameters to stimulate cell proliferation [27].

The ability of plasma to selectively deactivate without imparting detectable toxicity or detrimental effects to the surroundings has also been noted by several investigators. Alekseev et al. studied the antiviral effect of nanosecond pulsed DBD on human corneal cells infected with herpes simplex virus type 1 (HSV-1) [13]. Exposure of 35-40 s resulted in deactivation of the HSV-1 virus, while also imparting no detectable toxicity or detrimental effects to human corneas [13]. Similar selectivity results have been found by Fridman et al., in which, a floating electrode DBD (FE-DBD) was used to sterilize human tissue without incurring any detectable physical damage [13, 28].

Besides being selective, different types of microbes can be inactivated as shown by Joshi et al. [29]. The same FE-DBD plasma was able to deactivate E. coli, S. aureus, and multidrug-resistant S. aureus (MRSA) in respective biofilm and planktonic forms [29]. Filipić et al. reported that, ‘cold plasmas’ have demonstrated the capability to inactivate several types of viruses [30]. Capsid protein damage, nucleic acid disintegration and changes in lipid components were reported as primary disinfection mechanisms, and the primary RONS responsible were identified as ¹O₂, O₃, H₂O₂, ONOOH, ONOO⁻ and NOₓ. Plasma discharge in humid environment serves as a source of charged species and free radicals, notably the OH radical and other reactive oxygen and nitrogen species (RONS), which are detrimental to bacteria, viruses, and other microorganisms. [30, 39]

Enhancing the production of RONS can be the route towards increasing inactivation efficiency. Several studies have investigated whether the presence of water in plasma discharge generated more RONS. NTGP in the presence of moisture is known to produce several RONS that include N, O, OH, NO, NO₂ and possibly more [31]. Hanbal et al. studied the influence of DBD jet with air containing admixtures of water on Tobacco Mosaic viruses (TMV) [32]. It was observed that HNO₂, NO₂— and H₂O₂ produced in the plasma irradiation degraded nucleic acids in TMV resulting in viral collapse to subunits. Muranyi et al. studied the effect of synthetic air with 0-80% relative humidity (RH) as a discharge medium in a cascaded DBD on two strains of bacteria; they found that for A. niger, the bacterial mortality was the highest at 70% RH, whereas in case of B. subtilis, addition of any degree of RH deteriorated the mortality rate. The increased mortality of A. niger was attributed to the increased production of OH, which oxidizes the unsaturated fatty acids and proteins in the bacterial spores. The reduction in B. subtilis deactivation is attributed to the loss in discharge homogeneity of CDBD with increasing RH. Optimum deactivation of E. coli occurred at 43% RH when applying RF discharge in air [33]. In contrast to dry conditions, G. stearothermophilus spores showed no growth after indirect exposure to NTGP [34]. The reactions of N₂O₅ with H₂O and H₂O₂, forming HOONO and HOONO₂, both of which have virucidal properties are stated to be responsible for the enhanced deactivation of calicivirus and S. Heidelberg when treated with different plasma sources [35].

Even though literature highlights the effectiveness of non-thermal plasmas in disinfection, two major uphill challenges are preventing their widespread application in hospitals and other health-care facilities. A lack of knowledge of the physico-chemical processes that involves multitude of charged species and excited neutrals [28] and most DBD apparatus presented in the literature lacks the portability as well as the requirement of introducing specified amount of water vapor to incorporate the optimum RH.

Hence, the presently disclosed innovation presents a relatively simple, portable, planar DBD device with an electrically insulated annular flow channel built through the planar powered electrode that can be used to disinfect targeted areas and personal protective equipment (PPE), such as surgical masks, N95s, and respirator cartridges. The present disclosure explores the processes that influences the inactivation process specific to Escherichia (E.) coli and Bacillus (B.) atrophaeus and assesses the effectivity of misty plasma systems.

### SUMMARY OF THE PRESENTLY DISCLOSED SUBJECT MATTER

Broadly speaking, the presently disclosed subject matter relates to application of dielectric barrier discharge in misty plasma systems for disinfection of surfaces and equipment in health settings.

Presently disclosed subject matter also relates to system and corresponding and/or associated methodology for planar dielectric barrier discharge (DBD) device technology.

More particularly, presently disclosed technology relates to a DBD device with an electrically insulated annular flow channel built through the planar powered electrode.

The presently disclosed technology has higher effectivity than existing devices. While existing devices only use either UV radiation or air as the plasma discharge medium, the presently disclosed device also employs water vapor in a discharge medium, in addition to incorporating hydroxyl radicals in the discharge effluent, which are strong oxidative radicals along with the active discharge species produced from nitrogen and oxygen in air. Moreover, the device is portable and can be shifted among sections in a medical facility. Furthermore, the device is comparatively inexpensive to build and operate.

The device performs disinfection of surfaces via production of partially ionized gases called plasma. Existing plasma disinfection devices use air or sometimes helium gas as the discharge medium. We showed by using our device that igniting plasma in humid air, the disinfection efficacy can be increased manifolds. This is due to additional formation of hydroxide radicals that have stronger oxidative and thus disinfection properties that originate from plasma discharge in water vapor.

According to Grand View Research, the global ultraviolet disinfection equipment market size was valued at USD $7.13 billion in 2020 and is expected to grow at a compound annual growth rate (CAGR) of 12.1% over the forecast period, which is till 2028.

A system and corresponding and/or associated methodology is provided for planar dielectric barrier discharge (DBD) device technology. Per one exemplary embodiment, a DBD device is provided with an electrically insulated annular flow channel built through the planar powered electrode. The discharge medium is air saturated with water vapor that flows through the annulus and forms a stagnation discharge plane between the powered dielectric and the substrate. Application on Escherichia (E.) coli and Bacillus (B.) atrophaeus spores on agar and filter papers show that usage of a flowing humid discharge medium has a ˜40% higher efficacy than either a static or a dry flowing medium. The substrate turns acidic potentially due to the formation of reactive oxygen and nitrogen species. In the presence of water vapor, pH can be decreased further due to formation of hydrogen peroxide from recombination of OH radicals formed in the plasma.

Thus, approach of dielectric barrier discharge in water vapor promises to be efficient at disinfecting PPEs, which include soft surfaces, against SARS-CoV-2 or other coronaviruses which are not as resilient as the spores against which current tests have proved successful.

One presently disclosed exemplary embodiment relates to a system for disinfection of a target object including surfaces and equipment, comprising a planar electrode with an electrically insulated annular flow channel formed therethrough, with said channel having a respective input opening and output opening; an inlet tube carrying air saturated with water vapor into the annular flow channel input opening; and a pulsed DBD power supply. The power supply is preferably for controllably powering the planar electrode for a predetermined period of time to initiate dielectric barrier discharge (DBD) in a stagnation discharge plane formed between the output opening of the channel and a grounded target object, resulting in a flowing humid discharge medium from the planar electrode. Preferably, the discharge medium includes reactive oxygen and nitrogen species (RONS) for disinfecting effect on a target object.

Another presently disclosed exemplary embodiment relates to a portable misty plasma dielectric barrier discharge plasma system for disinfection of target objects. Such system preferably may comprise an air supply for producing an output of moisture saturated air; an electrode assembly comprising an electrode for receiving power, and axially situated between respective dielectric elements, with said electrode assembly forming an electrically insulated annular flow channel formed therethrough, with said channel having a respective input opening and output opening, with said input opening connected to the output of moisture saturated air from the air supply; and a pulsed dielectric barrier discharge (DBD) power supply for controllably powering the electrode for a predetermined period of time to initiate dielectric barrier discharge in a stagnation discharge plane formed between the output opening of the channel and a grounded target object, resulting in a flowing humid discharge medium from the electrode assembly including reactive oxygen and nitrogen species (RONS) for disinfecting effect on a target object.

It is to be understood from the complete disclosure herewith that the presently disclosed subject matter equally relates to both apparatus and corresponding and related methodology.

One presently disclosed exemplary methodology preferably relates to a method for disinfection of a target object including surfaces and equipment, comprising applying to the target object atmospheric pressure dielectric barrier discharge operating in a moisture saturated continuous airflow as the discharge medium.

Other example aspects of the present disclosure are directed to systems, apparatus, tangible, non-transitory computer-readable media, user interfaces, memory devices, and electronic smart devices or the like. To implement methodology and technology herewith, one or more processors may be provided, programmed to perform the steps and functions as called for by the presently disclosed subject matter, as will be understood by those of ordinary skill in the art.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the Figures or stated in the detailed description of such Figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

### BRIEF DESCRIPTION OF THE FIGURES

A full and enabling disclosure of the presently disclosed subject matter, including the best mode thereof, directed to one of ordinary skill in the art, is set forth in the specification, which makes reference to the appended Figures, in which:

FIG. 1(a) illustrates a schematic of an exemplary experimental setup for use in conjunction with presently disclosed subject matter;

FIG. 1(b) illustrates a voltage and current/time graph of an exemplary single characteristic voltage and current pulse in the discharge of a setup such as represented by present FIG. 1(a);

FIGS. 2(a) and 2(b) illustrate visualization of the discharge structure (a) without added humidity and (b) with added humidity, respectively;

FIG. 3(a) graphically illustrates comparison of bacterial load reduction for different discharge media for E. coli and B. atropaheus;

FIG. 3(b) graphically illustrates temporal evolution of bacterial load reduction for DBD in Air+H2O medium;

FIG. 4(a) graphically illustrates optical emission spectra of the discharge plane showing the OH and N2 2nd positive emission bands;

FIG. 4(b) graphically illustrates radial variation of pH from the agar center, post treatment;

FIG. 5(a) illustrates a side elevation view of an exemplary embodiment of an electrode configuration in accordance with presently disclosed subject matter;

FIG. 5(b) illustrates a cross-sectional view of an exemplary embodiment of an electrode configuration in accordance with presently disclosed subject matter as represented in FIG. 5(a);

FIGS. 6(a)-(d) illustrate respective petri dish colony results for E. coli treated for 2 minutes for (a) control, (b) static, (c) air, and (d) air+H2O respective medium conditions;

FIGS. 7(a)-7(d) illustrate respective petri dish colony results for B. atrophaeus treated for 20 mins for (a) control, (b) static, (c) air, and (d) air+H2O respective medium conditions; and

FIGS. 8(a) and 8(b) respectively visually illustrate DBD-treated strains of (a) E. coli and (b) B. atrophaeus, respectively, stained with SYBR green and PPI (proton pump inhibitors) to reveal dead DNA.

Repeat use of reference characters in the present specification and drawings is intended to represent the same or analogous features or elements or steps of the presently disclosed subject matter.

### DETAILED DESCRIPTION OF THE PRESENTLY DISCLOSED SUBJECT MATTER

Aspects and advantages of the presently disclosed subject matter will be set forth in part in the following description, or may be apparent from the description, or may be learned through practice of the presently disclosed subject matter.

It is to be understood by one of ordinary skill in the art that the present disclosure is a description of exemplary embodiments only, and is not intended as limiting the broader aspects of the disclosed subject matter. Each example is provided by way of explanation of the presently disclosed subject matter, not limitation of the presently disclosed subject matter. In fact, it will be apparent to those skilled in the art that various modifications and variations can be made in the presently disclosed subject matter without departing from the scope or spirit of the presently disclosed subject matter. For instance, features illustrated or described as part of one embodiment can be used with another embodiment to yield a still further embodiment. Thus, it is intended that the presently disclosed subject matter covers such modifications and variations as come within the scope of the appended claims and their equivalents.

The present disclosure is generally directed to system and corresponding and/or associated methodology for planar dielectric barrier discharge (DBD) device technology. More particularly, presently disclosed subject matter relates to a DBD device with an electrically insulated annular flow channel built through the planar powered electrode.

Atmospheric pressure dielectric barrier discharge operating in a moisture saturated continuous airflow as the discharge medium was studied as a prospective method for bacterial disinfection from soft surfaces. The effect on two different strains of bacteria: E. coli and B. atropaheus and for three different media for plasma discharge: static, airflow, moisture-saturated airflow was explored. Optical emission spectroscopy showed the generation of OH and reactive nitrogen species in the inter-electrode spacing between the dielectric and substrate for discharge in saturated air. The oxidizing ability of OH and H2O2 is primarily responsible for improved disinfection. The acidity of the agar medium was analyzed after treating for 25 mins. It was seen that for the case of moisture saturated air as the discharge medium, the pH change was observed for the longest radial distance from the point of influx. Compared to static conditions, the bacterial load reduction efficiency in moisture saturated air was found to be ˜1.5 and ˜2.5 times higher for E. coli and B. atrophaeus, respectively.

Experimental Method and Diagnostic Setup:

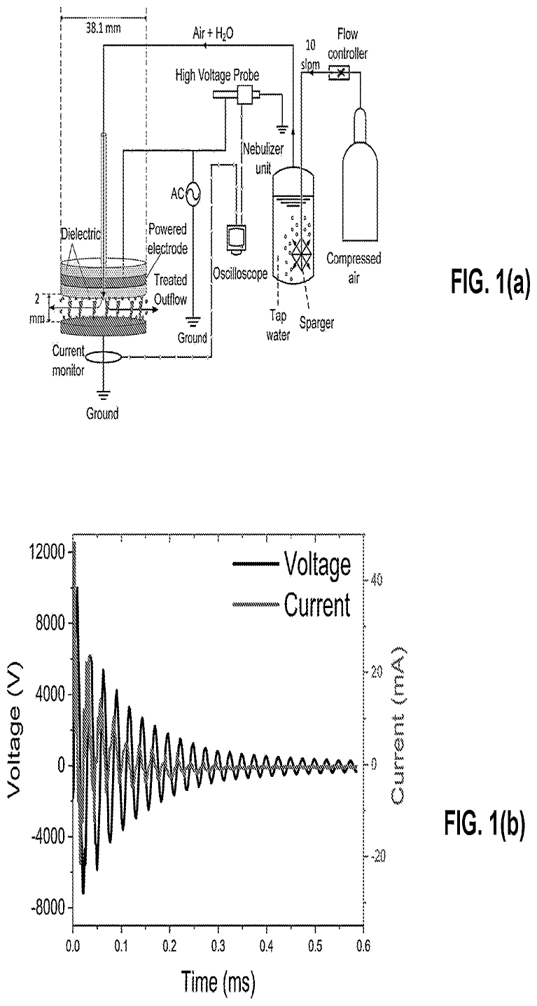

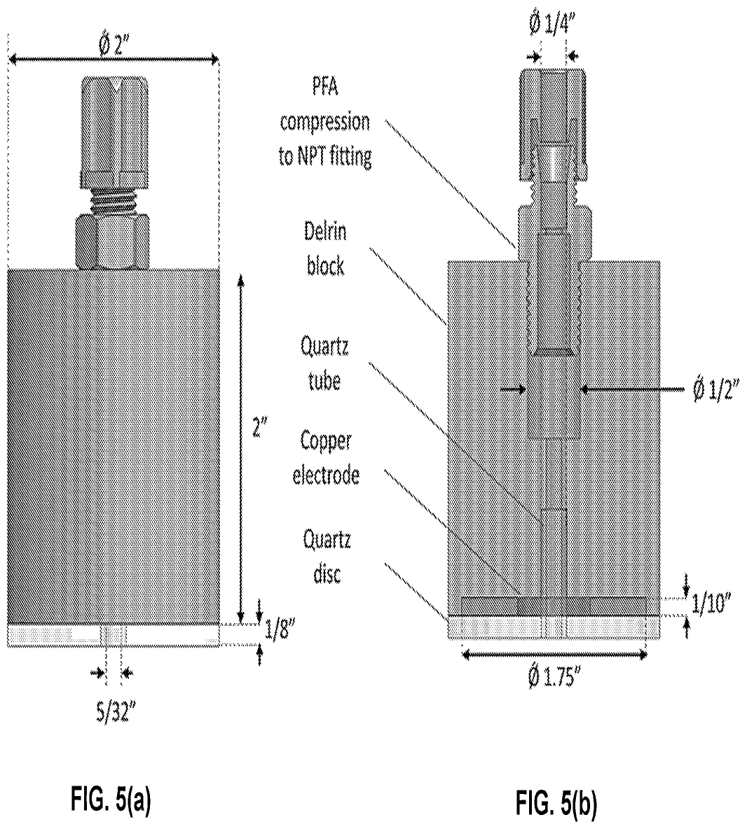

Possible exposure to personnel/users when disinfecting equipment or PPE utilizing misty plasma is not expected to cause any detrimental effects. Studies have shown that medical applications of plasma inhibit infections to human organs without introducing any detectable level of toxicity or denaturing of proteins [13]. Humid air was generated by passing compressed air through a bubble column containing filtered water, at 10 standard liters/min. FIG. 1(a) illustrates a schematic of an exemplary experimental setup for use in conjunction with presently disclosed subject matter. The saturated air was passed concentrically through an in-house designed electrode assembly (details of which are given in other information herein discussed in conjunction with subject FIGS. 5(a) and 5(b)). When static conditions were tested, the airflow was turned off. In case of studying airflow without any added water, an empty bubble column was used. The electrode assembly was positioned at 2 mm from the substrate, which, in turn, was placed on a ground plate. The substrate in the experimental setup consisted of agar media on which E. coli or B. atrophaeus had been plated. E. coli was treated in its ‘live’ form whereas B. atrophaeus was treated in its ‘sporulated’ form, i.e., these have thick spore coats protecting the bacterium until conditions become favorable for germination. This makes these bacilli highly resistant to conventional sterilization procedures [34, 36-38]. The discharge was initiated in the stagnation plane formed by the saturated air between the lower surface of the electrode (the dielectric itself) and the grounded substrate.

A regulated 15-25 kV, 50-4000 Hz, 300 W pulsed DBD power supply (Advanced Plasma Solutions) was used to initiate the DBD discharge. The DBD HV pulser was operated at 20 kV, 1670 Hz with a pulse-width of 0.3 ms. When Power_{R.M.S.} is 17.5 W, Power/area is 0.87 W/cm². An Agilent mixed-signal oscilloscope MS07054B (500 MHz, 4 GS/s) was used to measure the discharge voltage and discharge current. In particular, FIG. 1(b) illustrates a voltage and current/time graph of an exemplary single characteristic voltage and current pulse in the discharge of a setup such as represented by present FIG. 1(a).

The voltage at the copper electrode was measured via a North Star PVM-4 1000:1 HV probe and the discharge current was measured at the ground with a Pearson 6515 current monitor. The power density was maintained at 0.87 W/cm². The optical emissions spectrometer of the discharge is captured (FIG. 1(b)) with an integration time of 10 s by Ocean Optics HR4000 CG-UV-NIR with an optical fiber directed towards the center of the discharge plane.

A suspension of 10⁵ colony forming units (CFUs) per ml of E. coli (K-12 strain, ATCC 10798) and B. atrophaeus spores (BG-105, Crosstex Medical Company) were made separately in Luria-Bertani (LB, Fisher Scientific) and 1× phosphate-buffered saline (1×PBS), respectively. Enumeration of both E. coli and B. atrophaeus spores were done through optical density measurement at 600 nm (OD600) with a Biotek Synergy two microplate reader. Quantification was done using 2×10⁹ CFUs/OD600. 100 μl of the selected suspension containing 1×10⁴ CFUs was spread on solidified LB agar on 100×15mm petri dishes to a height of 0.5 cm. Each dish with either E. coli or B. atrophaeus was placed below the electrode such that the surface of the agar was 2 mm from the bottom plane of the dielectric. After the experiments, the petri dishes were incubated at 37° C. for 24 h to facilitate the growth of colonies. The colonies were later counted with Image J software.

Results and Discussion:

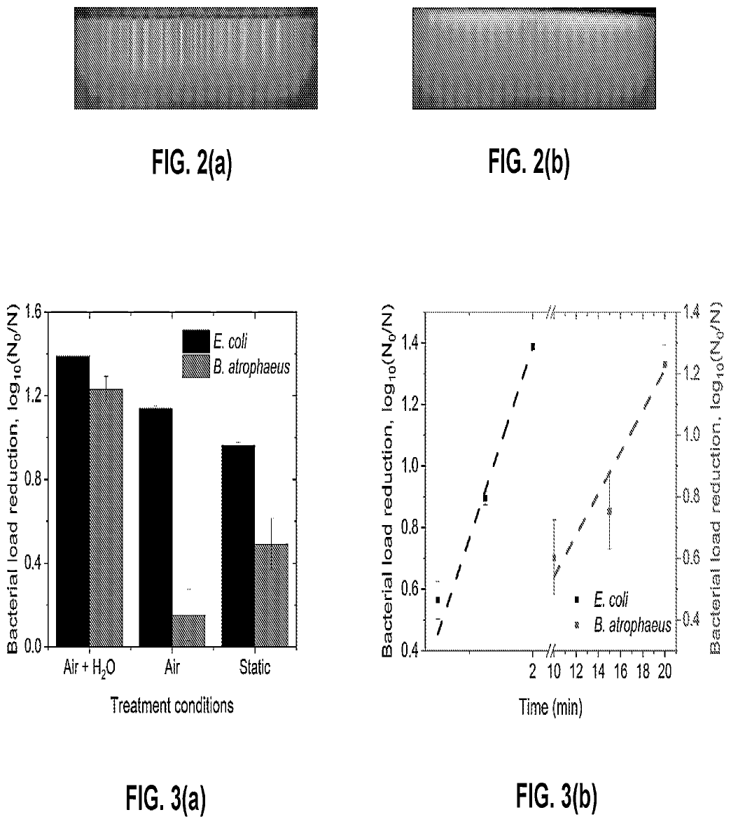

The dielectric barrier discharge (DBD) is initiated in the stagnation plane formed by the saturated air between the lower surface of the electrode (the dielectric itself) and the grounded substrate. FIGS. 2(a) and 2(b) illustrate visualization of the discharge structure (a) without added humidity and (b) with added humidity, respectively. In particular, FIG. 2(a) shows that without the influx of air or water vapor, the DBD discharge is non-uniform and filamentary in nature when compared to FIG. 2(b). In other words, as shown by FIG. 2(a)(no added humidity), the BDB discharge is nonuniform with visible streamers, while per FIG. 2(b)(air saturated with water vapor) it is uniform discharge with streamers almost nonexistent. The discharge emits the characteristic bluish color of nitrogen emissions. With the influx of moisture saturated air from the center of the top plane, the discharge becomes more uniform, and the filamentary nature is reduced, as represented per FIG. 2(b).

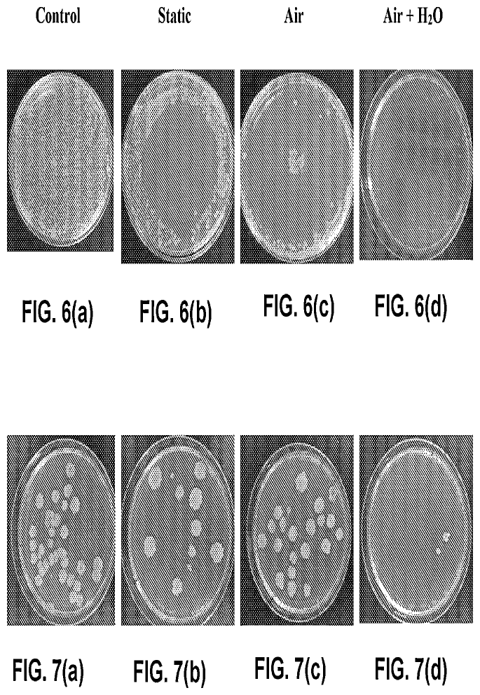

The experiments were conducted for three different discharge types: i) static ambient, ii) airflow with no water added at room temperature (298 K) and one atm and iii) airflow saturated with water vapor under the same conditions. Multiple samples were treated at each condition to reduce uncertainty. Visual observation (FIGS. 6(a)-6(d) and 7(a)-7(d)) for E. coli and B. atropheus) showed that after the same treatment duration, the petri dishes that were treated with moisture-saturated air shows the least number of colonies.

FIG. 3(a) graphically illustrates comparison of bacterial load reduction for different discharge media for E. coli and B. atropaheus. FIG. 3(b) graphically illustrates temporal evolution of bacterial load reduction for DBD in Air+H₂O medium.

Since B. atrophaeus was treated in its sporulated form, it offered a greater resistance to RONS from the plasma generation and needed to be treated for a longer duration. It is observed that while, for E. coli, it was sufficient to treat for 2 mins to obtain maximum disinfection, B. atrophaeus did not show any noticeable effect of disinfection until the treatment time reached greater than 10 mins. The deactivation efficiency is calculated by a logarithmic reduction method described in [39] as log₁₀(N₀/N), where No is the bacterial count in the control samples that did not undergo any treatment and N is the bacterial count after undergoing DBD treatment. FIG. 3(a) compares the inactivation extent for the three treatment conditions. Adding humid airflow to the discharge medium increases the deactivation efficacy by ˜1.5 and ˜2.5 times for E. coli and B. atrophaeus, respectively. This agrees with the visual observations showing the superiority of the moisture-laden airflow as the discharge medium.

The effect of treatment durations was also assessed. E. coli were treated for 0.5, 1.25 and 2 min and B. atrophaeus petri dishes were treated for durations of 10, 15, and 20 minutes. FIG. 3(b) shows the dependence of bacterial load reduction on treatment times for only the moisture-saturated air as the discharge medium. It is observed that for both the bacterial strains, the bacterial load reduction increases in a linear correlation with treatment time. FIG. 3(b) also confirms that for B. atrophaeus, the minimum treatment time for noticeable bacterial load reduction is 5 times that is required for E. coli due to its spore-protection form.

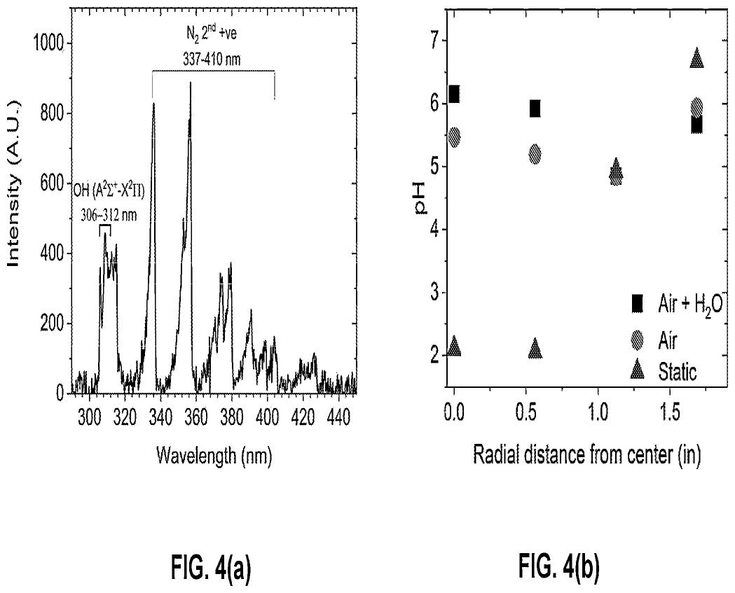

FIG. 4(a) graphically illustrates optical emission spectra of the discharge plane showing the OH and N₂ 2nd positive emission bands. FIG. 4(b) graphically illustrates radial variation of pH from the agar center, post treatment.

For both B. atrophaeus spores and E. coli colonies, the DBD in humid medium proved to be of the highest effectiveness. Stated another way, bacterial load reduction for the three discharge media studied shows in case of both strains of the bacteria, dielectric barrier discharge (DBD) in saturated air medium has the highest effectiveness. The effectiveness could be due to OH radicals, since they are strong oxidizing agents that react with cell membranes, denature the nucleic lipids and proteins in the bacterial cells, and thus deactivate the bacteria. This may be attributed to the higher concentration of OH, H, O and H₂O₂ being generated from DBD discharge in the presence of a greater degree of water.

Optical emission spectra of the discharge show the presence of OH(A-X) band at 306-312 nm [40] and reactive nitrogen species (N₂ 2nd+ve, 337-410 nm [41]) in the discharge, as represented per FIG. 4(a). Direct reliable, accurate quantification of OH and other reactive species is beyond the scope of the present work.

OH radicals are highly reactive and the combination of two OH radicals produced in plasma discharge to form one molecule of H₂O₂ in the afterglow can be regarded as a possible decay mechanism of OH. Since H₂O₂ is acidic, its formation is supposed to lower the pH. Thus, pH measurement of the treated medium is adopted as an indirect method to assess treatment effectiveness. The agar medium is treated for 25 mins with all of the three discharge mediums and the radial variation of pH from the center of influx is measured using Horiba LAQUAtwin pH-22 Compact Meter. FIG. 4(b) shows that the static conditions induce the largest pH drop but with the smallest treatment range, i.e., localized, since it had no induced flow and thus no convective species transport. Despite DBD in both ‘air’ and ‘air+H₂O’ having comparable treatment ranges, air+H₂O, induces a slightly larger pH drop farthest away from the center. The drop in pH of the medium in static and dry airflow conditions may be attributed to low-oxidizing radicals and neutrals that do not primarily originate from the presence of water.

Furthermore, in further experiments (not shown here), it is seen that presence of O₂ enhances the production of H₂O₂ in plasma NTP discharge in presence of H₂O. This may explain why DBD in static conditions was not able to considerably deactivate B. atrophaeus spores despite lowering the pH by the greatest degree. The increase in acidity of the medium in presence of water vapor can be potentially due to the formation and subsequent dissociation of H₂O₂, formation of peroxynitric acid (HOONO₂₆) and peroxynitrous acid (HOONO), all of which have bactericidal properties [35] but may not drastically decrease pH since these are not direct deposited on the substrate due to being associated with a radial convective flow. A sharp drop in pH might also be undesirable for medical applications.

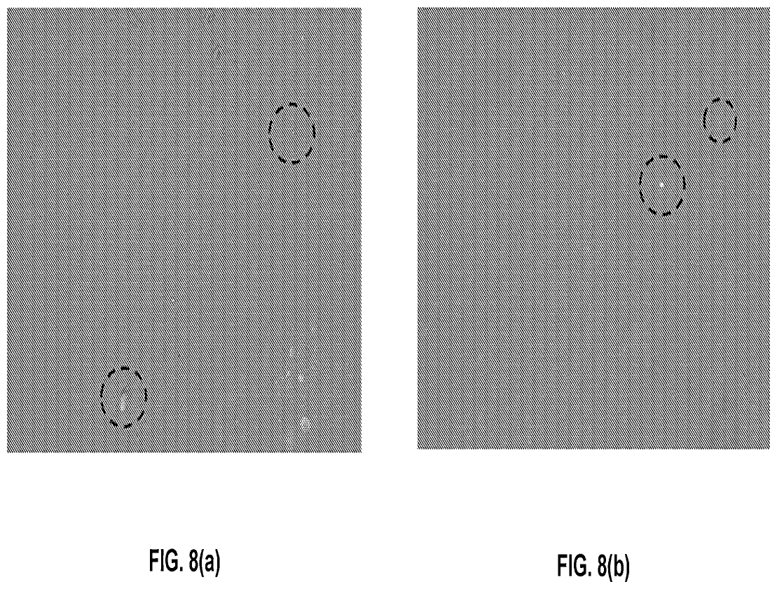

To analyze the cause of the deactivation of the treated stains, live/dead experiments were conducted on the treated samples with SYBR green and subsequently with propidium iodide (PI). SYBR green can penetrate both live and dead cells whereas PI can penetrate only dead cells. In other words, Propidium iodide (PI) can only stain DNA of dead cells; i.e. PI cannot enter a live cell. When the SYBR green and PI stains are merged, the resultant dead cells are stained as yellow. FIGS. 8(a) and 8(b) respectively visually illustrate DBD-treated strains of (a) E. coli and (b) B. atrophaeus, respectively, stained with SYBR green and PPI (proton pump inhibitors) to reveal dead DNA. The images in FIGS. 8(a) and 8(b) show yellow stained (dotted line encircled) cells for both E. coli and B. atrophaeus highlighting that the cells have been ‘killed’ by the DBD plasma, i.e., which confirms the disinfection capability of DBD.

Limitations and Future Directions:

In this work, the DBD setup is portable, is able to disinfect sporulated bacillus, which are typically highly resistant to disinfectants, and does so within a short time period. Based on the efficacy, it could be said with reasonable confidence that it will be a successful tool against viruses as well. Furthermore, an agar surface was treated which can be classified as soft and has ramifications for disinfecting surfaces of PPEs, mask airways, tubing, often touched locations such as arms of sofas/chairs, patient stirrups, straps if present and the mist can penetrate hard to reach areas such as in-between computer keyboards or behind monitors. The approach of DBD in water vapor promises to be efficient at disinfecting items such as PPEs enabling quick, safe, and efficient re-use. Furthermore, the fact that DBD discharge in presence of water vapor promotes maximum deactivation efficacy without incurring any significant change in the pH of the medium is promising and the involved processes need further investigation.

FIG. 5(a) illustrates a side elevation view of an exemplary embodiment of an electrode configuration in accordance with presently disclosed subject matter. FIG. 5(b) illustrates a cross-sectional view of an exemplary embodiment of an electrode configuration in accordance with presently disclosed subject matter as represented in FIG. 5(a). The electrode includes a circular sandblasted copper ring (OD 1.75″, ID 0.39″, thickness: 0.06″), which is powered and is fused to the top surface of a circular quartz ring (OD 2″, ID 0.25″, thickness: 0.125″), serving as the dielectric. The entire setup is housed in a Delrin (highly-crystalline engineered thermoplastic) block (OD 2″, thickness: 2″). The inner diameter of the Delrin cylindrical block is drilled to 0 0.25″ from the bottom to a height of 0.25″ and to a 0 0.5″ at the top to a depth of 1″. The housing connects to an inlet PFA (perfluoroalkoxy) tube carrying the air saturated with water vapor. At the bottom of the housing, a quartz tube (OD 0.25″, ID 0.16″) is fitted and its walls are fused with adhesive to the inner walls of the dielectric. This tube serves as the passage for the saturated air to the ground to form the discharge stagnation plane.

FIGS. 6(a)-6(d) illustrate respective petri dish colony results for E. coli treated for 2 minutes for (a) control, (b) static, (c) air, and (d) air+H2O respective medium conditions. Similarly, FIGS. 7(a)-7(d) illustrate respective petri dish colony results but instead for B. atrophaeus treated for 20 mins for (a) control, (b) static, (c) air, and (d) air+H2O respective medium conditions. FIGS. 8(a) and 8(b) respectively visually illustrate DBD-treated strains of (a) E. coli and (b) B. atrophaeus, respectively, stained with SYBR green and PPI (proton pump inhibitors) to reveal dead DNA. The dotted line encircled features showing stained (yellow) cells highlight that such (a) E. coli and (b) B. atrophaeus cells, respectively, have been ‘killed’ by the presently disclosed DBD plasma technology.

Further summarizing, disinfecting capability of DBD in three different discharge media: static air, air flow, and air flow saturated with water vapor, were studied and disinfection efficacy of DBD in water vapor saturated air flow is found to be most effective. Radial variation of pH indicates that along with transport effects, reactive oxygen and hydroxide species originating from water vapor, possibly due to formation and subsequent dissociation of H₂O₂, are also responsible for the enhanced treatment. Colorimetry test shows the presence of dead DNA from the treated bacteria, confirming the disinfection capability of DBD. Future studies could involve spectroscopic measurements to identify the reactive species produced in the discharge in air+H₂O medium, scanning electron microscope images of the treated strains and applications on viruses, namely, bacteriophages.

While certain embodiments of the disclosed subject matter have been described using specific terms, such description is for illustrative purposes only, and it is to be understood that changes and variations may be made without departing from the spirit or scope of the subject matter.

### References

ADDIN EN.REFLIST 1. Li, Y., R. Atif, K. Chen, J. Cheng, Q. Chen, Z. Qiao, G. Fridman, A. Fridman, and H.-F. Ji, Polymerization of d-ribose in dielectric barrier discharge plasma. Plasma, 2018. 1(1): p. 144-149.

2. Na, Y. H., G. Park, E. H. Choi, and H. S. Uhm, Effects of the physical parameters of a microwave plasma jet on the inactivation of fungal spores. Thin solid films, 2013. 547: p. 125-131.

3. Laurita, R., A. Miserocchi, M. Ghetti, M. Gherardi, A. Stancampiano, V. Purpura, D. Melandri, P. Minghetti, E. Bondioli, and V. Colombo, Cold atmospheric plasma treatment of infected skin tissue: evaluation of sterility, viability, and integrity. IEEE Transactions on Radiation and Plasma Medical Sciences, 2017. 1(3): p. 275-279.

4. Yan, D., J. H. Sherman, and M. Keidar, Cold atmospheric plasma, a novel promising anti-cancer treatment modality. Oncotarget, 2017. 8(9): p. 15977.

5. Laroussi, M., M. Kong, G. Morfill, and W. Stolz, Plasma medicine: applications of low-temperature gas plasmas in medicine and biology. 2012: Cambridge University Press.

6. Ercan, U. K., S. S. Joshi, A. Yost, S. O'Toole, M. Paff, E. Melchior, S. G. Joshi, and N. Gogotsi, Inhibition of biofilms by non-thermal plasma treated novel solutions. Advances in Microbiology, 2014. 4(16): p. 1188.

7. Fridman, G., M. Peddinghaus, M. Balasubramanian, H. Ayan, A. Fridman, A. Gutsol, and A. Brooks, Blood coagulation and living tissue sterilization by floating-electrode dielectric barrier discharge in air. Plasma Chemistry and plasma processing, 2006. 26(4): p. 425-442.

8. Kalghatgi, S. U., G. Fridman, M. Cooper, G. Nagaraj, M. Peddinghaus, M. Balasubramanian, V. N. Vasilets, A. F. Gutsol, A. Fridman, and G. Friedman, Mechanism of blood coagulation by nonthermal atmospheric pressure dielectric barrier discharge plasma. IEEE Transactions on plasma science, 2007. 35(5): p. 1559-1566.

9. Kalghatgi, S., G. Friedman, A. Fridman, and A. M. Clyne, Endothelial cell proliferation is enhanced by low dose non-thermal plasma through fibroblast growth factor-2 release. Annals of biomedical engineering, 2010. 38(3): p. 748-757.

10. Coulombe, S., V. Léveillé, S. Yonson, and R. Leask, Miniature atmospheric pressure glow discharge torch (APGD-t) for local biomedical applications. Pure and Applied Chemistry, 2006. 78(6): p. 1147-1156.

11. Shekhter, A. B., V. A. Serezhenkov, T. G. Rudenko, A. V. Pekshev, and A. F. Vanin, Beneficial effect of gaseous nitric oxide on the healing of skin wounds. Nitric oxide, 2005. 12(4): p. 210-219.

12. Smith, J. B., I. Adams, and H.-F. Ji, Biomolecule response to nonthermal plasma. Plasma Medicine, 2017. 7(4).

13. Alekseev, O., K. Donovan, V. Limonnik, and J. Azizkhan-Clifford, Nonthermal dielectric barrier discharge (DBD) plasma suppresses herpes simplex virus type 1 (HSV-1) replication in conical epithelium. Translational vision science & technology, 2014. 3(2): p. 2-2.

14. Lloyd, G., G. Friedman, S. Jafri, G. Schultz, A. Fridman, and K. Harding, Gas plasma: medical uses and developments in wound care. Plasma Processes and Polymers, 2010. 7(3-4): p. 194-211.

15. Laroussi, M., I. Alexeff, and W. L. Kang, Biological decontamination by nonthermal plasmas. IEEE Transactions on Plasma Science, 2000. 28(1): p. 184-188.

16. Hague, M., M. Sartelli, J. McKimm, and M. A. Bakar, Health care-associated infections—an overview. Infection and drug resistance, 2018. 11: p. 2321.

17. Jinadatha, C., R. Quezada, T. W. Huber, J. B. Williams, J E Zeber, and L. A. Copeland, Evaluation of a pulsed-xenon ultraviolet room disinfection device for impact on contamination levels of methicillin-resistant Staphylococcus aureus. Bmc Infectious Diseases, 2014. 14: p. 7.

18. Penno, K., R. A. Jandarov, and M. M. Sopirala, Effect of automated ultraviolet C-emitting device on decontamination of hospital rooms with and without real-time observation of terminal room disinfection. American Journal of Infection Control, 2017. 45(11): p. 1208-1213.

19. Rutala, W. A. and D. J. Weber, Best practices for disinfection of noncritical environmental surfaces and equipment in health care facilities: A bundle approach. American Journal of Infection Control, 2019. 47: p. A96-A105.

20. Carling, P. C., M. F. Parry, and S. M. Von Beheren, Identifying opportunities to enhance environmental cleaning in 23 acute care hospitals. Infect Control Hosp Epidemiol, 2008. 29(1): p. 1-7.

21. Doll, M., M. Stevens, and G. Bearman, Environmental cleaning and disinfection of patient areas. International Journal of Infectious Diseases, 2018. 67: p. 52-57.

22. Rutala, W. A. and D. J. Weber, Are Room Decontamination Units Needed to Prevent Transmission of Environmental Pathogens? Infection Control & Hospital Epidemiology, 2015. 32(8): p. 743-747.

23. Rutala, W. A., D. J. Weber, and HICPAC, Guideline for Disinfection and Sterilization in Healthcare Facilities, 2008, update May 2019, Centers for Disease Control and Prevention

24. Hong, Y., J. Kang, H. Lee, H. Uhm, E. Moon, and Y. Park, Sterilization effect of atmospheric plasma on Escherichia coli and Bacillus subtilis endospores. Letters in applied microbiology, 2009. 48(1): p. 33-37.

25. Laroussi, M., C. Tendero, X. Lu, S. Alla, W. L. J. P. P. Hynes, and Polymers, Inactivation of bacteria by the plasma pencil. 2006. 3(6-7): p. 470-473.

26. Shi, X.-M., G.-J. Zhang, X.-L. Wu, Z.-Y. Peng, Z.-H. Zhang, X.-J. Shao, and Z.-S. Chang, Effect of low-temperature plasma on deactivation of hepatitis B virus. IEEE Transactions on Plasma Science, 2012. 40(10): p. 2711-2716.

27. Kalghatgi, S., C. M. Kelly, E. Cerchar, B. Torabi, O. Alekseev, A. Fridman, G. Friedman, and J. Azizkhan-Clifford, Effects of non-thermal plasma on mammalian cells. PloS one, 2011. 6(1): p. e16270.

28. Fridman, G., G. Friedman, A. Gutsol, A. B. Shekhter, V. N. Vasilets, and A. Fridman, Applied plasma medicine. Plasma processes and polymers, 2008. 5(6): p. 503-533.

29. Joshi, S. G., M. Paff, G. Friedman, G. Fridman, A. Fridman, and A. D. Brooks, Control of methicillin-resistant Staphylococcus aureus in planktonic form and biofilms: a biocidal efficacy study of nonthermal dielectric-barrier discharge plasma. American journal of infection control, 2010. 38(4): p. 293-301.

30. Filipić, A., I. Gutierrez-Aguirre, G. Prime, M. Mozetiĉ, and D. Dobnik, Cold plasma, a new hope in the field of virus inactivation. Trends in Biotechnology, 2020. 38(11): p. 1278-1291.

31. Wilson, A., D. Staack, T. Farouk, A. Gutsol, A. Fridman, and B. Farouk, Self-rotating dc atmospheric-pressure discharge over a water-surface electrode: regimes of operation. Plasma Sources Science and Technology, 2008. 17(4): p. 045001.

32. Hanbal, S. E., K. Takashima, S. Miyashita, S. Ando, K. Ito, M. M. Elsharkawy, T. Kaneko, and H. Takahashi, Atmospheric-pressure plasma irradiation can disrupt tobacco mosaic virus particles and RNAs to inactivate their infectivity. Archives of virology, 2018. 163(10): p. 2835-2840.

33. Maeda, Y., N. Igura, M. Shimoda, and I. J. A. b. Hayakawa, Inactivation of Escherichia coli K12 using atmospheric gas plasma produced from humidified working gas. 2003. 23(4): p. 389-395.

34. Venezia, R. A., M. Orrico, E. Houston, S.-M. Yin, and Y. Y. Naumova, Lethal activity of nonthermal plasma sterilization against microorganisms. Infection Control & Hospital Epidemiology, 2008. 29(5): p. 430-436.

35. Moldgy, A., G. Nayak, H. A. Aboubakr, S. M. Goyal, and P. J. Bruggeman, Inactivation of virus and bacteria using cold atmospheric pressure air plasmas and the role of reactive nitrogen species. Journal of Physics D: Applied Physics, 2020. 53(43): p. 434004.

36. Schinkothe, J., H. A. Scheinemann, S. Diederich, H. Freese, M. Eschbaumer, J. P. Teifke, and S. Reiche, Airborne Disinfection by Dry Fogging Efficiently Inactivates Severe Acute Respiratory Syndrome Coronavirus 2 (SARS-CoV-2), Mycobacteria, and Bacterial Spores and Shows Limitations of Commercial Spore Carriers. Applied and Environmental Microbiology, 2021. 87(3): p. e02019-20.

37. Rattanakul, S. and K. Oguma, Inactivation kinetics and efficiencies of UV-LEDs against Pseudomonas aeruginosa, Legionella pneumophila, and surrogate microorganisms. Water research, 2018. 130: p. 31-37.

38. Zhang, C., Y. Li, C. Wang, and X. Zheng, Different inactivation behaviors and mechanisms of representative pathogens (Escherichia coli bacteria, human adenoviruses and Bacillus subtilis spores) in g-C3N4-based metal free visible-light-enabled photocatalytic disinfection. Science of The Total Environment, 2021. 755: p. 142588.

39. Gershman, S., M. B. Harreguy, S. Yatom, Y. Raitses, P. Efthimion, and G. Haspel, A low power flexible dielectric barrier discharge disinfects surfaces and improves the action of hydrogen peroxide. Scientific reports, 2021. 11(1): p. 1-12.

40. Qazi, H., Q.-Y. Nie, H.-P. Li, X.-F. Zhang, and C.-Y. Bao, Comparison of electrical and optical characteristics in gas-phase and gas-liquid phase discharges. Physics of Plasmas, 2015. 22(12): p. 123512.

41. Georgescu, N., C. P. Lungu, A. R. Lupu, and M. Osiac, Atomic oxygen maximization in high-voltage pulsed cold atmospheric plasma jets. IEEE transactions on plasma science, 2010. 38(11): p. 3156-3162.

What is claimed is:

1. Method for disinfection of a target object including surfaces and equipment, comprising applying to the target object atmospheric pressure dielectric barrier discharge operating in a moisture saturated continuous airflow as the discharge medium.

2. The method according to claim 1, wherein applying comprises using a planar dielectric barrier discharge (DBD) device having a planar powered electrode with an electrically insulated annular flow channel formed therethrough.

3. The method according to claim 1, wherein the discharge medium is air saturated with water vapor that flows through the annular flow channel to form a stagnation discharge plane between the powered electrode and the target object, and resulting in a flowing humid discharge medium from the powered electrode.

4. The method according to claim 2, wherein:
the annular flow channel has a respective input opening and output opening;
the planar powered electrode includes a powered dielectric adjacent the annular flow channel output opening; and
dielectric barrier discharge (DBD) is initiated in a stagnation plane formed by the saturated air between a surface of the dielectric and a grounded target object.

5. The method according to claim 3, further comprising:
implementing a portable system for producing the discharge medium; and
using the discharge medium for disinfecting at least one of surfaces of personal protective equipment (PPE), mask airways, tubing, furniture, patient stirrups, straps, electronic apparatus, computer keyboards or monitors.

6. The method according to claim 2, wherein the planar powered electrode comprises a copper electrode received between an associated quartz tube and a quartz disc, and with the copper electrode and quartz tube at least partially surrounded by a thermoplastic housing.

7. The method according to claim 6, wherein:
the annular flow channel has a respective input opening and output opening; and
the thermoplastic housing is connected to an inlet tube carrying air saturated with water vapor into the annular flow channel input opening.

8. The method according to claim 7, further comprising:
controllably powering the copper electrode with a pulsed DBD power supply for a predetermined period of time used to initiate DBD discharge; and
wherein the quartz tube serves as a passage for saturated air to a grounded target object to form a discharge stagnation plane whenever DBD discharge is initiated.

9. The method according to claim 8, wherein the predetermined period of time is in a range of from about 2 minutes to about 10 minutes.

10. The method according to claim 8, wherein the predetermined period of time is at least 10 minutes.

11. The method according to claim 8, wherein the pulsed DBD power supply comprises a regulated 15-25 kV, 50-4000 Hz, 300 W pulsed DBD power supply used to initiate the DBD discharge.

12. The method according to claim 11, further comprising operating the pulsed DBD power supply with a pulse-width of less than 1.0 ms and so as to maintain a power density of at least 0.5 W/cm².

13. The method according to claim 1, wherein the discharge medium includes reactive oxygen and nitrogen species (RONS) for disinfecting effect on a target object.

14. System for disinfection of a target object including surfaces and equipment, comprising:
a planar electrode with an electrically insulated annular flow channel formed therethrough, with said channel having a respective input opening and output opening;
an inlet tube carrying air saturated with water vapor into the annular flow channel input opening; and
a pulsed DBD power supply for controllably powering the planar electrode for a predetermined period of time to initiate dielectric barrier discharge (DBD) in a stagnation discharge plane formed between the output opening of the channel and a grounded target object, resulting in a flowing humid discharge medium from the planar electrode, wherein the discharge medium includes reactive oxygen and nitrogen species (RONS) for disinfecting effect on a target object.

15. The system according to claim 14, wherein:
the planar electrode includes a powered dielectric adjacent the annular flow channel output opening; and
the dielectric barrier discharge (DBD) is initiated in the stagnation plane formed by the saturated air between a surface of the dielectric and a grounded target object.

16. The system according to claim 14, wherein:
the system comprises a portable system for producing the discharge medium; and
the target object comprises at least one of surfaces of personal protective equipment (PPE), mask airways, tubing, furniture, patient stirrups, straps, electronic apparatus, computer keyboards or monitors.

17. The system according to claim 15, wherein the planar electrode comprises a copper electrode received between an associated quartz tube and a quartz disc, and with the copper electrode and quartz tube at least partially surrounded by a thermoplastic housing.

18. The system according to claim 14, wherein the predetermined period of time is in a range of from about 2 minutes to about 10 minutes.

19. The system according to claim 14, wherein the predetermined period of time is at least 10 minutes.

20. The system according to claim 14, wherein the pulsed DBD power supply comprises a regulated 15-25 kV, 50-4000 Hz, 300 W pulsed DBD power supply used to initiate the DBD discharge and operated so as to maintain a power density of at least 0.5 W/cm².

21. A portable misty plasma dielectric barrier discharge plasma system for disinfection of target objects, the system comprising:
an air supply for producing an output of moisture saturated air;
an electrode assembly comprising an electrode for receiving power, and axially situated between respective dielectric elements, with said electrode assembly forming an electrically insulated annular flow channel formed therethrough, with said channel having a respective input opening and output opening, with said input opening connected to the output of moisture saturated air from the air supply; and
a pulsed dielectric barrier discharge (DBD) power supply for controllably powering the electrode for a predetermined period of time to initiate dielectric barrier discharge in a stagnation discharge plane formed between the output opening of the channel and a grounded target object, resulting in a flowing humid discharge medium from the electrode assembly including reactive oxygen and nitrogen species (RONS) for disinfecting effect on a target object.

22. The system according to claim 21, wherein the electrode comprises a copper electrode received between an associated quartz tube and a quartz disc, and with the copper electrode and quartz tube at least partially surrounded by a thermoplastic housing.

23. The system according to claim 21, wherein the predetermined period of time is in a range of from about 2 minutes to at least 10 minutes.

24. The system according to claim 23, wherein the pulsed DBD power supply comprises a regulated 15-25 kV, 50-4000 Hz, 300 W pulsed DBD power supply used to initiate the dielectric barrier discharge and operated so as to maintain a power density of at least 0.5 W/cm².
